(19) United States
(12) Patent Application Publication
(10) Pub. No.: US 2003/0204156 A1
(43) Pub. Date: Oct. 30, 2003

(54) Knee brace with patella stabilizer

(76) Inventors: Ronald E. Nelson, Cambridge, MN (US); Curt F. Mueller, Prairie du Sac, WI (US)

Correspondence Address: FOLEY & LARDNER, 150 EAST GILMAN STREET, P.O. BOX 1497, MADISON, WI, 53701-1497, US

(21) Appl. No.: 10/133,033

(22) Filed: Apr. 26, 2002

(51) Int. Cl. A61F 5/00; A61F 5/37

(52) U.S. Cl. 602/26

(57) ABSTRACT

A knee brace for use by athletes or others requiring protection and support of the knee. The knee brace protects against abnormal motions of the knee, and provides direct and indirect patella stabilization. A base comprised of a tubular sleeve of elastic material is configured to closely fit around the knee joint and adjacent upper and lower leg portions. One or more generally upright lateral pockets containing semi-rigid stays are provided on the sides of the base for lateral support. Direct patella support is provided by a reinforced circular hole in the base and two straps that cross the front of the leg above the knee. Indirect patella support is provided by a resilient member in a pocket. Different resilient members and a lower support strap located across the resilient member just below the patella may be used to adjust the amount of indirect patella support provided by the resilient member.

### FIELD OF THE INVENTION

This invention relates generally to the field of articles worn by persons to reduce the likelihood, severity, or exacerbation of injury to the body, and more specifically to the field of braces worn on the knee.

### BACKGROUND OF THE INVENTION

Flexible knee braces are used by athletes and other persons engaged in vigorous physical activity to protect the knee from injury and to avoid exacerbation of existing injury. The knee is one of the most heavily used joints of the body, as it is used in any activity that involves walking or running. The knee is also a common subject of injury, due to the relatively high levels of stress it must bear under dynamic loads that are often multiples of the entire weight of the body. During normal ambulation, in occupations involving physical labor, and especially during strenuous sports, the knee can undergo abnormal motions as a result of quick changes in direction, fatigue, uneven surfaces, or impacts. These abnormal motions can cause sprains or more serious injuries, such as dislocation, stretching, or tearing of the tissues that make up the knee.

Several different types of abnormal motion can cause injury to the knee. First, hyperextension of the knee joint can occur, wherein the knee flexes in its normal front to back fashion but beyond its normal range of motion. A second type of abnormal motion is axial rotation, wherein the lower leg is twisted rotationally relative to the thigh about the knee joint. A third type of abnormal motion is lateral flexure of the lower leg relative to the thigh, wherein the knee joint flexes from side to side instead of the normal front to back motion. In addition, abnormal motion of the patella (kneecap) can result in injuries such as chondromalacia patella, which is a softening or degeneration of the undersurface of the patella, and dislocation of the patella, also known as subluxation of the patella.

Devices to protect the knee against abnormal motions have been used for many years, in a variety of specific embodiments which vary in their abilities to protect against the different types of abnormal motions. Besides protecting the knee against abnormal motions, the devices sometimes provide additional benefits such as insulating the knee to keep it warm, protecting the knee against impact, or compressing the knee to reduce discomfort. However, the protections afforded by these devices against abnormal motion are often accompanied by a reduction in range or ease of normal motion. These devices can also have other undesirable aspects such as added weight on the leg, potential for self-injury or injury to others caused by rigid components, difficulty of application and removal, cost, appearance, irritation or chafing of the skin, and other drawbacks.

Knee pads comprised of simple elastic sleeves worn on the leg with padding in front of the knee are well known in the prior art. Such knee pads are commonly used in sports that vigorously stress the knee such as volleyball and soccer, and also in occupations such as carpet laying or floor tile work. Knee pads do not protect against abnormal motion or otherwise support the knee, instead they merely protect the knee against injuries caused by impact or repeated contact with hard surfaces.

Elastic wraps are also known in the prior art. In its simplest form, such a wrap might consist of a single elastic bandage wrapped about the knee. The prior art also contains examples of more complicated knee wraps with multiple support straps wrapped about the knee in a variety of ways, referred to herein as “pure wrap” devices. For example, U.S. Pat. No. 5,873,848 discloses a complex spiral wrapping structure including no rigid or semi-rigid elements which is meant to provide a flexible orthopedic brace supporting the patella or other portion of the human body. Another example is found in U.S. Pat. No. 6,142,965, which discloses a complex spiral wrapping structure with spiral wraps in two directions, also without rigid or semi-rigid elements. Knee wraps of this type are typically lightweight and relatively inexpensive to manufacture. They can provide compression or pressure to the patella or knee area to protect against dislocation of the patella, and they can also provide some support against abnormal axial rotational motion. However, this type of knee wrap is not able to protect against hyperextension or against abnormal lateral flexure.

The prior art also contains devices that build on the pure wrap approach by utilizing a sleeve structure, referred to herein as “sleeve-based” devices. The sleeve structure is typically implemented as a tubular sleeve of elastic material, or as a reclosable sleeve which may be fastened about the knee area. Devices implemented as a tubular sleeve must typically be manufactured in a range of sizes to accommodate knees of varying dimensions, increasing their cost to manufacture and distribute, while reclosable sleeves can often be manufactured in one or two sizes and still fit the majority of people.

Examples of the sleeve-based approach are found in U.S. Pat. No. 6,063,048 which discloses a tubular elastic sleeve with a support strap meant to be comfortable and inexpensive to manufacture, U.S. Pat. No. 5,925,010 which discloses a tubular elastic sleeve meant to provide support and therapeutic heating without moisture buildup, and U.S. Pat. Nos. 5,728,057 and 5,728,058 which disclose two forms of an elastic knee wrap built upon a reclosable sleeve that is meant to provide positionable compression and therapeutic heating. In common with the pure wrap devices, sleeve-based devices provide compression, but they lack the ability to protect against hyperextension or abnormal lateral flexure. Unlike the pure wrap devices, these devices are not meant to protect against abnormal axial rotation, but they do provide therapeutic heating and they may be easier to apply than complex wrap structures.

A third class of devices to protect the knee is based on mechanical hinges, herein referred to as “hinge-based” devices. One example of this approach is found in U.S. Pat. No. 4,726,362 which discloses a knee brace comprised of a composite hinge assembly with three pivot points attached to the leg using upper and lower leg pads or wraps, meant to protect against several forms of abnormal injury-causing motions. Additional examples are found in U.S. Pat. Nos. 4,573,455 and 4,844,057, which disclose composite hinge mechanisms with three pivot points intended to follow the complex motion of the normal knee while preventing abnormal motions, and in U.S. Pat. Nos. 5,514,082 and 5,514,083, which disclose knee braces containing upper and lower leg pads and a hinge assembly with a single pivot point meant to protect against abnormal anterior tibial movement (hyperextension), to aid in patellar tracking and alignment, to treat a disorder common to adolescent males known as Osgood Schlatter's Disease (OSD), and to have other advantages such as light weight and low cost. Although these hinge-based devices can provide good protection against abnormal motions such as hyperextension, axial rotation and lateral flexure, they typically will not protect against impact to the knee joint or dislocation of the patella, nor do they provide compression or insulation. Notwithstanding efforts made to reduce the cost of hinge-based devices, the machining required in their construction is relatively expensive compared to structures which do not require machining. Hinge-based devices are often relatively heavy because of the metal or other rigid parts they contain, and also bulky because of the clearance required for the moving parts that make up the hinge.

Examples of a different form of hinged-based device are found in a series of patents disclosing various improvements to an orthopedic brace invention first disclosed in U.S. Pat. No. 3,902,482. For example, U.S. Pat. No. 5,562,605 discloses a complex hinged brace structure intended to treat injuries to the medial collateral ligament, and also to treat unicompartmental osteoarthritis, which is a mechanical malfunction of the knee that results in excessive and uneven wear of the knee joint. Another example is found in U.S. Pat. No. 5,797,864, which discloses a similar brace with the addition of features meant to stabilize the position of the patella. Braces of this form have a relatively high cost of manufacture due to their construction as well as the other disadvantages of hinge-based braces, such as excessive bulk and weight. Further, this form of brace is intended mainly for remedial treatment of existing injury, and not for prevention of injury or use during athletic activity.

Another class of devices is specifically meant to stabilize the patella, herein called “patella-stabilizing” devices. These devices may use various means to apply direct pressure to the patella, or they may apply pressure to the patella tendon to indirectly force the patella into proper alignment, or they may use a combination of the direct and indirect approaches.

Examples of direct patella-stabilizing devices are found in U.S. Pat. No. 5,221,252, which discloses a releasable elastic sleeve with a reinforced circular opening for the patella that is meant to provide direct and adjustable compressive force on the patella, and in U.S. Pat. No. 5,411,037 which discloses a tubular elastic sleeve with a semi-circular tension member placed in the curve of the knee cap that is meant to exert direct medial pressure to center the knee cap. Self-adhesive bandages meant to provide direct patella support are also found in the prior art, as disclosed in U.S. Pat. Nos. 5,944,682 and 5,711,312. More complicated approaches to providing direct patella support are found in U.S. Pat. Nos. 4,296,744, 5,613,943, and 5,807,298 which disclose various forms of a knee brace with a floating patella pad meant to maintain effective patella support throughout a range of knee motions.

An example of an indirect patella-stabilizing device is found in U.S. Pat. No. 4,334,528, which discloses a knee strap meant to press against the patella tendon to force the patella into proper alignment. Because the patella tendon is relatively immobile compared to the patella itself, pressure applied to the patella tendon is meant to provide better stabilization of the patella compared to the devices which apply direct pressure to the patella itself.

Other devices, as exemplified in U.S. Pat. Nos. 6,077,242, 6,080,124, 5,417,646, and 5,865,782, provide patella support through a combination of direct pressure to the patella and indirect pressure via the patella tendon. Although these devices may provide protection against injuries related to abnormal motion of the patella, they do not provide protection against other abnormal motions of the knee such as hyperextension, rotation, or lateral flexure.

Another class of devices combines a reclosable sleeve or wraparound structure with semi-rigid stays, herein called stay-based devices. The stays used in these devices are flexible in some directions, but relatively rigid in other directions. As applied, the stays typically provide support and limit movement of the knee to within the normal range. In addition to the stays, these devices sometimes contain a direct patella stabilizing element.

One example of a pure stay-based device which does not include specific elements for patella support is found in U.S. Pat. No. 5,513,658, which discloses a reclosable elastic sleeve with side stays, meant to warm and protect the knee without restricting normal motion or trapping moisture.

Other stay-based devices do provide direct patella support using various means. For example, crossed straps may be used for this purpose, as in U.S. Pat. No. 4,366,813 which discloses a tubular elastic sleeve with upright semi-rigid stays and crossed support straps, meant to protect the knee against abnormal lateral and rotational motion and to provide direct patella support for the knee cap to prevent hyperextension and dislocation. Reinforced circular openings and buttresses in various shapes are also used to provide direct patella support, as shown in U.S. Pat. Nos. 5,399,153, 5,656,023, 5,759,167, 5,823,981, and 5,865,777. These devices can provide protection against abnormal motions of the knee such as hyperextension, rotation, and lateral flexure as well as limited protection against dislocation of the patella.

Each of the various forms of knee brace has disadvantages. Pure wrap devices provide only direct patella support, and they do not protect against hyperextension or lateral flexure. Sleeve-based devices have these same disadvantages, plus devices based on tubular elastic sleeves provide even less protection against rotation, they must be manufactured in a variety of sizes, and they may be difficult to apply. Hinge-based devices are typically heavy, bulky, and expensive to manufacture. Because of the rigid components in hinged-base devices, they can present a hazard to participants in contact sports. Although some hinged-based devices can provide patellar support, this is generally done using direct pressure, with its disadvantages. Prior patella stabilizing devices, both of the direct pressure and the superior indirect pressure type, generally do not provide protection against the other forms of abnormal knee motion, such as hyperextension, rotation, or lateral flexure. Stay-based devices can provide good protection against abnormal knee motion, but such devices protect the patella using direct pressure, with its disadvantages.

### SUMMARY OF THE INVENTION

A knee brace according to the present invention can protect against all forms of abnormal knee motion and provide indirect patella support, while avoiding undue restriction of movement or bunching. It can also provide therapeutic warming without undue moisture buildup. It is easy to adjust, fasten, and remove, and it is usable by a wide range of people with a variety of knee problems and knee sizes.

The present invention includes a base which may be worn in snug covering relationship to the knee including the kneecap, patella tendon, and adjacent portions of the leg of a person. Preferably, this base may be an openable elastic sleeve which may be fastened about the knee and adjacent portions of the leg, but it may also be a tube made of an elastic material which may be slipped onto the knee and adjacent portions of the leg. The base preferably includes an opening at the rear to avoid bunching or undue restriction of movement, but this is not required. The base is preferably made of an elastic material which provides generalized support and compression to the knee area, along with therapeutic warming, but other materials may be used. Hook and loop fastener material is preferably used to fasten the base about the knee, allowing the tightness and position of the base to be adjusted without removing it entirely, and allowing the base to be manufactured in a single size which can be adjusted to fit a wide range of people, but other fasteners such as buckles, clasps, buttons, or pins may be used. All such alternative embodiments will be referred to herein as a base.

One or more upright support members are attached to the sides of the base, to provide support and protect the knee against abnormal motions. Preferably, these upright support members may be semi-rigid stays made of compressed spring structures, but they may also be semi-rigid stays made of resilient plastic or similar material. The upright support elements may also be rigid hinges, or they may be a combination of semi-rigid stays and hinges.

The base includes a resilient member to apply pressure to the patella tendon to force the patella into proper alignment using the indirect form of patella stabilization. Because the patella tendon is relatively immobile compared to the patella itself, this indirect form of patella stabilization using pressure applied to the patella tendon can provide better stabilization of the patella compared to devices that use only direct pressure to the patella itself.

Preferably, the resilient member may be a cylindrical structure having a circular cross section, but it may also be non-cylindrical and have, for example, a rectangular, ovoid, or triangular cross section. The resilient member may preferably be made of a flexible hollow plastic tube, but it may also be made of a solid but compressible foam material or other materials with similar properties. The degree of pressure applied to the patella tendon may be adjusted using resilient members having differing levels of resilience. This may preferably be accomplished using flexible hollow plastic tubes of varying diameters, but it may also be accomplished using materials having varying resilience, such as different plastics or compressible foam materials. The amount of pressure applied by the resilient member on the patella tendon can also be adjusted using a separate strap that is provided below the knee. The indirect form of patella stabilization can be disabled entirely by removing the resilient member completely, if that feature is not required by a particular user of the brace.

The knee brace of the invention preferably incorporates a circular opening for the patella and crossed straps to provide direct patella stabilization. The tension of the cross straps can be adjusted independently to vary the amount of direct pressure applied to the patella itself. In addition to providing direct patella stabilization, the circular opening locates the brace with respect to the patella and enhances the beneficial indirect patella stabilization provided by the resilient member by maintaining the resilient member in an optimal position on the patella tendon.

Further objects, features, and advantages of the invention will be apparent from the following detailed description when taken in conjunction with the accompanying drawings.

### BRIEF DESCRIPTION OF THE DRAWINGS

In the drawings:

FIG. 1 is a perspective view of the front and side of a knee brace according to the invention fitted on the leg of a person, with the support straps attached;

FIG. 2 is a perspective view of the front and side of a knee brace according to the invention, like that of FIG. 1, but with the support straps in an open or unattached position;

FIG. 3 is a side elevation view of a knee brace according to the invention, showing the side opposite that shown in FIG. 1;

FIG. 4 is a front view of a knee brace according to the invention laid flat and with the support straps in an open or unattached position.

FIG. 5 is a rear view of a knee brace according to the invention laid flat and with the support straps in an open or unattached position; and

FIG. 6 is a cross-sectional view of the knee brace of FIG. 5 taken along the line 6-6 thereof.

FIG. 7 is a cross-sectional view taken generally along the lines 7-7 of FIG. 4.

FIG. 8 is a front elevation view of an upright support element that is a semi-rigid stay made of a compressed spring structure.

FIG. 9 is a side elevation view of an upright support element that is a semi-rigid stay made of a compressed spring structure.

FIG. 10 is a side elevation view of an upright support element that is a hinge.

FIG. 11 is a front elevation view of an upright support element that is a hinge.

FIG. 12 is a perspective view of an upright support element that is a hinge.

### DETAILED DESCRIPTION OF THE INVENTION

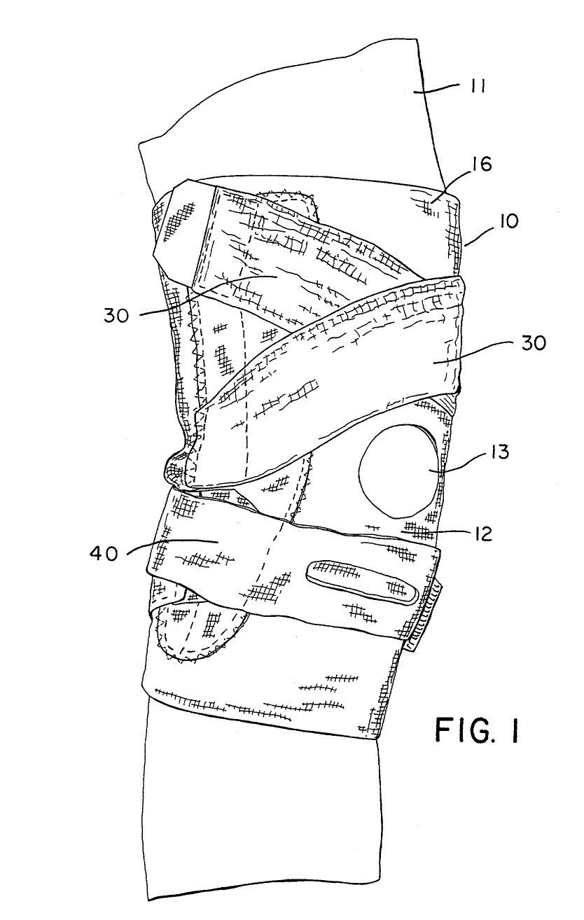

Referring to the drawings, FIGS. 1-3 show a knee brace indicated generally at 10 worn on the leg 11 of a person. The knee brace 10 includes a base 12 which is preferably formed of a reclosable sleeve made from a sheet of elastic material. The base 12 may also be formed, for example, of a tubular elastic sleeve shaped to fit snugly about the knee and adjacent leg portions. The base 12 may include a hole 13 which generally matches the size of the patella to align and support the patella, and to aid in positioning the knee brace in proper position with respect to the patella. The front surface 16 of the sheet of elastic material may be partially or entirely covered in loop type fastener material.

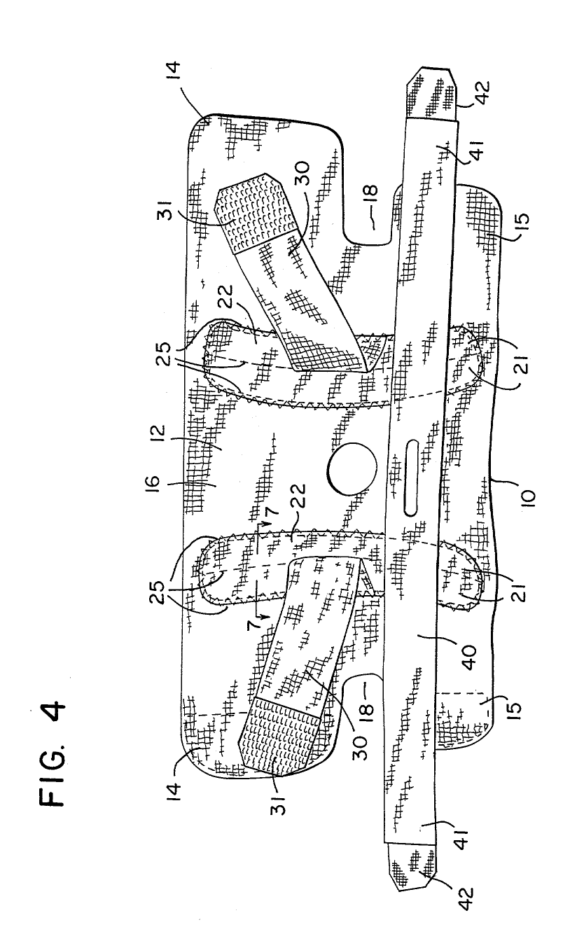

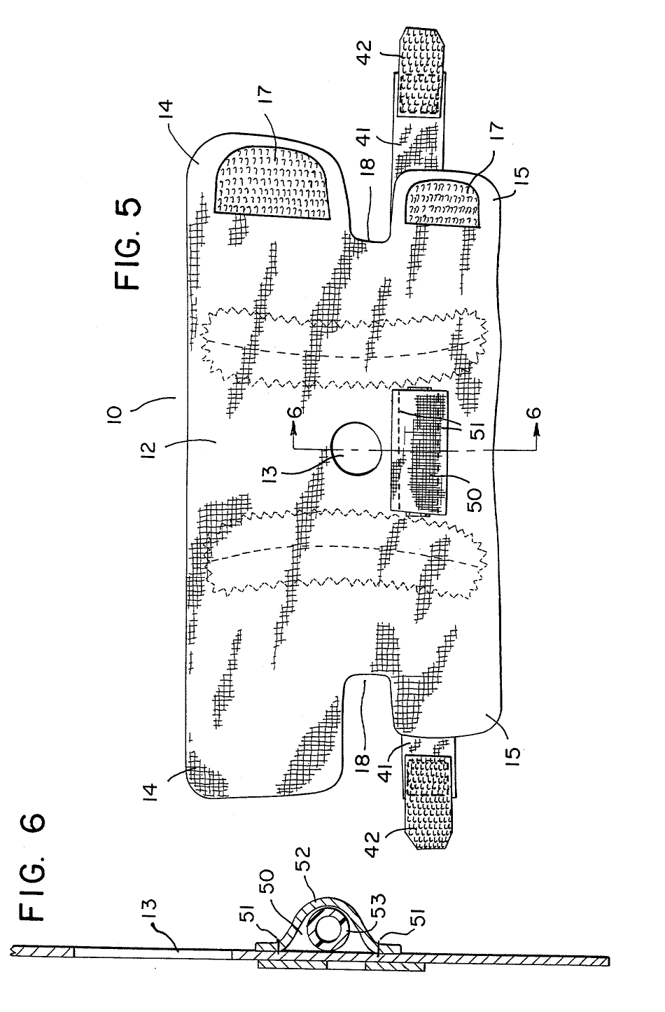

As best shown in FIGS. 4 and 5, the sides of the base 12 terminate in upper fastening tabs 14 and lower fastening tabs 15. As shown in FIGS. 4 and 5, the base may have at least one opening 18 on a side of the base such that an opening is formed at the rear to prevent bunching or undue restriction of motion when the knee brace is worn on the leg of a person. As shown in FIG. 5, areas of hook type fastening material 17 are sewn, or otherwise fixed, onto one of the upper fastening tabs 14 and one of the lower fastening tabs 15. When the knee brace 10 is fitted upon the leg of a person such that the hole 13 is placed on the patella so that the patella extends at least partially through the hole, the upper fastening tabs 14 and lower fastening tabs 15 can be wrapped about the leg with the areas of hook type fastening material 17 overlapping and engaging the loop type fastening material on the front surface 16, such that the brace is secured above the knee by the upper fastening tabs 14 and below the knee by the lower fastening tabs 15. A gap 18 is provided between the upper fastening tabs 14 and the lower fastening tabs 15, so that when the knee brace 10 is fitted upon the leg, an opening at the rear of the knee is formed.

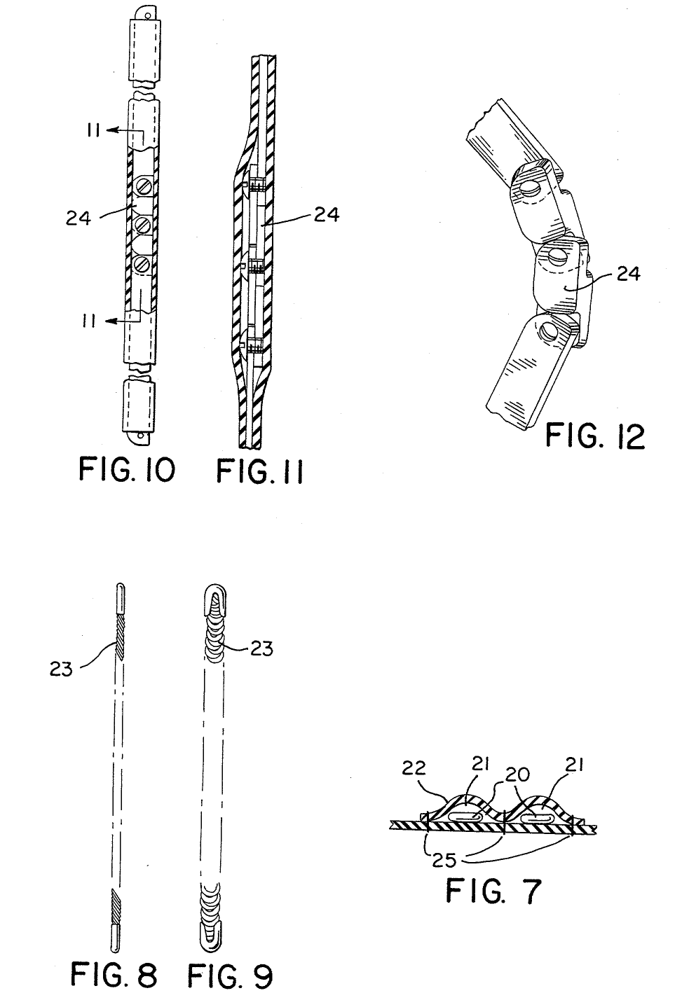

Upright support members 20 (shown in cross-section in FIG. 7) are placed in elongated side pockets 21. The elongated side pockets 21 are formed between vertical sewn seams 25 that fix the side pocket cover strips 22 to the base 12. The side pocket cover strips 22 may be made of the same elastic sheet material as the base 12 of the knee brace 10, and the front surface of the side pocket cover strips 22 is preferably also covered with loop type fastener material. The upright support members 20 are preferably semi-rigid stays 23, as shown in FIGS. 8 and 9, which may be made of a flattened spiral core of stainless steel or other flexible material of conventional construction commonly used in various types of braces. The upright support members 20 may also be a hinge 24 which is formed of at least two pivotally connected links, examples of which are shown in FIGS. 10, 11, and 12 which illustrate a preferred hinge having four links 24A, 24B, 24C, and 24D. There may be a single elongated side pocket with only one upright support member, or there may be one or more elongated side pockets on each side of the knee with an upright support member in each elongated side pocket. The elongated side pockets may be openable at one end to allow removal of the upright support members, so that the brace may be washed or so that different upright support members may be inserted to adjust the amount and type of support provided.

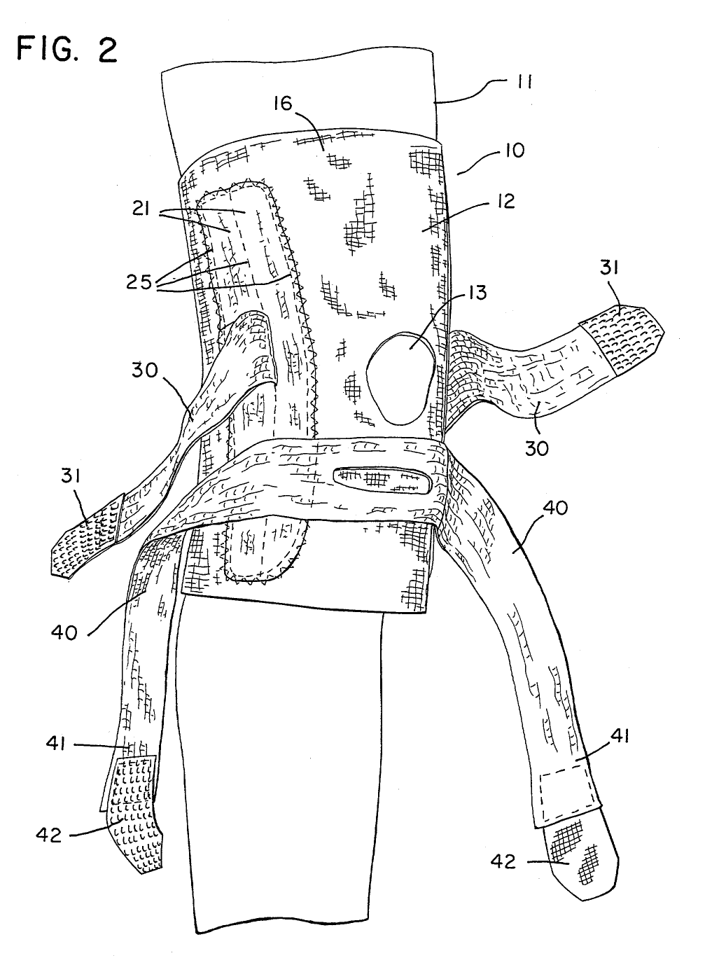

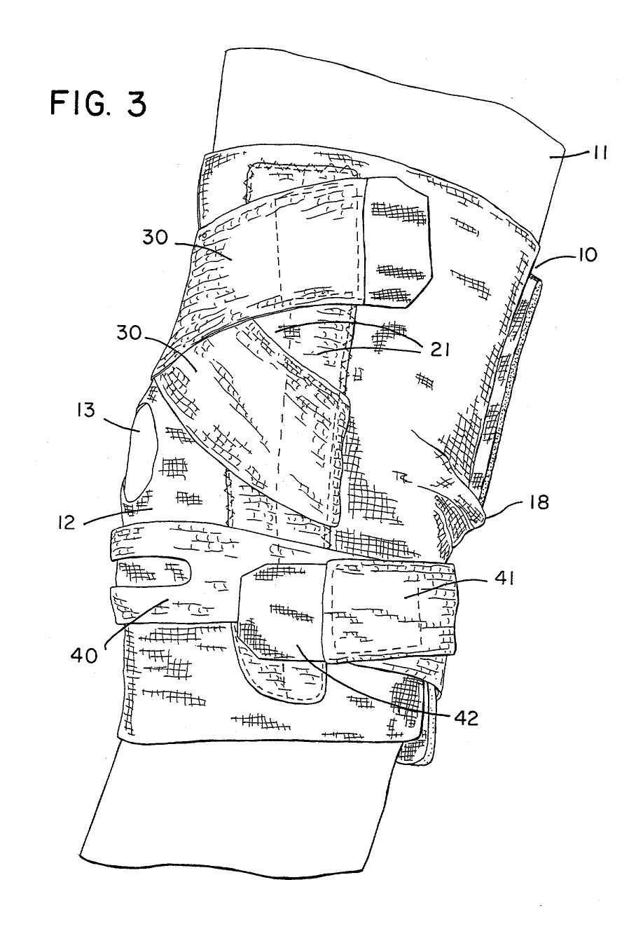

As shown in FIGS. 1-4, upper support straps 30 are sewn to the base 12. The upper support straps 30 may preferably be sewn to the base 12 and the side pocket cover strips 22 using the same seams 25 used to secure the side pocket cover strips 22 to the base 12, or the upper support straps 30 may be sewn to the base using separate seams. An area of hook type fastener material 31 is sewn or otherwise fixed onto the inside surface of the free ends of upper support straps 30, as shown in FIGS. 2 and 4. Once the upper and lower fastening tabs 14 and 15 and the areas of hook type fastening material 17 have been used to fasten the knee brace 10 to the leg, the free ends of the upper support straps 30 may be fastened in a crossed fashion around the front of the upper leg, as illustrated in FIGS. 1 and 3, by pressing the hook type material 31 on the free ends of the upper support straps 30 onto the front surfaces of the base 12 or the side pocket cover strips 22.

As shown most clearly in FIGS. 2 and 4, a lower support strap 40 having free ends 41 is also sewn to the base 12. The lower support strap 40 may preferably be made of the same elastic sheet material as the base 12 of the knee brace 10, and the front surface of the lower support strap 40 is also preferably covered with loop type fastener material. As shown most clearly in FIGS. 2 and 5, an area of hook type fastener material 42 is sewn or otherwise fixed to the free ends 41 of the lower support strap 40. Once the upper and lower fastening tabs 14 and 15 and the areas of hook type fastening material 17 have been used to fasten the knee brace 10 to the leg, the free ends 41 of the lower support strap 40 are extended around the lower leg, as shown in FIGS. 1 and 3, and secured by pressing the hook type material 42 on the free ends 41 of the lower support strap 40 onto the front surfaces 16 of the base 12, the side pocket cover strips 22, or the lower support strap 40.

As shown in FIGS. 5 and 6, an elongated inside pocket 50 is secured to the base 12. The inside pocket 50 may be formed between horizontal sewn seams 51 that fix an inside pocket cover strip 52 to the inside surface of the base 12 just below the hole 13 at approximately the same vertical location at which the lower support strap is sewn to the outside of the elastic base 12. The inside pocket cover strip 52 may be made of the same elastic sheet material as the base 12 of the knee brace 10. A resilient member 53 may be inserted in the pocket 50 to provide additional support and alignment to the patella. The resilient member 53 may preferably be a cylindrical structure having a circular cross section such as a piece of hollow flexible plastic tubing of a length substantially matching the length of the inside pocket 50, but it may also be non-cylindrical and have, for example, a rectangular, ovoid, or triangular cross section. Although preferably the resilient member 53 may be made of a flexible hollow plastic tube, it may also be made of a solid but compressible foam material or other materials with similar properties. The resilient member 53 applies pressure resiliently against the patella ligament, and may also apply pressure to the bottom of the patella.

The degree of pressure applied to the patella tendon may be adjusted by substituting a resilient member 53 having a different level of resilience. Preferably, this may be accomplished using flexible hollow plastic tubes of varying diameters, but it may also be accomplished using materials having varying resilience, such as different plastics or compressible foam materials. The amount of pressure applied by the resilient member 53 to the patella tendon can be adjusted using a separate strap that is provided below the knee. The indirect form of patella stabilization can also be disabled entirely by removing the resilient member 53 completely, if that feature is not required by a particular user of the brace.

Although hook and loop type fastener material is preferably used to fasten the brace about the knee and to secure the upper and lower support straps, equivalent fasteners such as clasps, buckles, pins, or buttons may be substituted for the hook and loop type fastener material.

It is understood that the invention is not confined to the embodiments set forth herein as illustrative, but embraces all such forms thereof that come within the scope of the following claims.

What is claimed is:

1. A knee brace, comprising:
(a) a base wearable in snug covering relationship to a knee including the kneecap, patella tendon, and adjacent portions of a leg of a person;
(b) at least one upright support member mounted to the base; and
(c) a resilient member mounted to the base just below the kneecap of the person such that pressure is applied to the patella tendon by the resilient member to aid in the alignment of the kneecap when the base is worn in snug covering relationship to the knee, the kneecap, and adjacent portions of the leg.

2. The knee brace of claim 1 wherein each upright support member is a semi-rigid stay.

3. The knee brace of claim 1 wherein each upright support member is a hinge comprising at least two links pivotally connected together and mounted to the base at a position to the side of a knee when the knee brace is in position on the leg of the person.

4. The knee brace of claim 1 wherein the base is a tubular sleeve.

5. The knee brace of claim 1 wherein the base is an openable and reclosable sleeve.

6. The knee brace of claim 1 wherein the base includes an opening located at the position of the patella that is approximately the same size as the patella.

7. The knee brace of claim 1 wherein the resilient member is removable.

8. The knee brace of claim 1 wherein the resiliency of the resilient member may be adjusted.

9. The knee brace of claim 1 wherein the upright support member is removable.

10. The knee brace of claim 1 further comprising support straps affixed to the base at one end thereof and fastenable across the upper portion of the leg.

11. The knee brace of claim 1 further comprising a support strap fastenable around the lower portion of the leg at approximately the same vertical location as the resilient member.

12. The knee brace of claim 1 wherein the resilient member comprises a hollow flexible tubing.

13. The knee brace of claim 12 including a pocket on the base just below the kneecap of the person and wherein the flexible tubing is removably mounted in the pocket.

14. The knee brace of claim 1 including a pocket on the base just below the kneecap of the person and wherein the resilient member is removably mounted in the pocket.

15. The knee brace of claim 1 including at least one opening on the side of the base such that an opening is formed at the rear of the knee when the knee brace is in position on the leg of the person.
